(19) United States
(12) Patent Application Publication
(10) Pub. No.: US 2020/0270580 A1
(43) Pub. Date: Aug. 27, 2020

(54) METHOD FOR PREPARING AGONIST FOR IMPROVING BOAR SPERM MOTILITY

(71) Applicant: Qingdao Agricultural University, Qingdao (CN)

(72) Inventors: Huansheng DONG, Qingdao (CN); Jinqiang CUI, Qingdao (CN); Qingjie PAN, Qingdao (CN); Jiaojiao HUANG, Qingdao (CN)

(21) Appl. No.: 16/656,598

(22) Filed: Oct. 18, 2019

(30) Foreign Application Priority Data
Feb. 22, 2019 (CN) 201910140331.8

(51) Int. Cl. C12N 5/071; A01N 1/02

(52) U.S. Cl. CPC C12N 5/0683; A01N 1/0284; C12N 2501/73; C12N 2501/734; A61B 10/0058; C12N 2500/38; C12N 2501/998; C12N 2500/02; C12N 2500/32

(57) ABSTRACT

A method for preparing an agonist for improving boar sperm motility includes: (1) collecting and infiltrating a testicular tissue of a young boar 3-5 days after born; (2) washing the tissue with PBS; (3) incubating the tissue in a cell culture medium, and centrifuging the cell suspension to remove a supernatant; (4) adding hyaluronidase and collagenase IV followed by shaking, and adding the cell culture medium and centrifuging to remove a supernatant; (5) adding trypsin and deoxyribonuclease followed by shaking, and adding the cell culture medium; (6) filtering the cell suspension by a cell sieve; (7) centrifuging to remove a supernatant, and adding the cell culture medium; (8) culturing and passaging the cells; (9) culturing and storing the cell suspension. The invention has the advantages of good application effect, low cost and simple production process.

### CROSS-REFERENCE TO RELATED APPLICATIONS

This application claims the benefit of priority from Chinese Patent Application No. 201910140331.8, filed on Feb. 22, 2019. The content of the aforementioned application, including any intervening amendments thereto, is incorporated herein by reference in its entirety.

### TECHNICAL FIELD

This application relates to biotechnology, and more specifically to a method for preparing an agonist for improving boar sperm motility.

### BACKGROUND OF THE INVENTION

The quality monitoring for boar sperms plays a vital role in the daily management of boars, since the sperm quality is directly related to the breeding conception rate and litter size, and in turn affects the economic benefits of the piggery. After collected, the boar semen is subjected to examination, which helps understand the status of the boar sperms and determine the reproductive performance of the boar. Routine examination for sperms generally includes the amount, odor, color, motility and density of semen, in which the sperm motility is an important and well-recognized indicator for characterizing the quality of boar sperms. Sperm motility refers to the percentage of mobile sperms in the semen, and since only the mobile sperms may have normal viability and fertilization ability, the sperm motility is closely concerned with the female conception rate. Therefore, the sperm motility is generally used as a primary indicator in routine examination to evaluate the quality of semen. Given the above, if the sperm motility can be improved effectively, the breeding rate of breeding pigs will be accordingly promoted, increasing the economic benefits of the piggery. Therefore, it is of great significance to prepare an agonist capable of significantly improving the sperm motility of boars.

Currently, the methods for improving the sperm motility of boars mainly include: (1) intramuscular injection with testosterone, where the injection is performed once a day for three consecutive days; (2) artificial feeding with ASCOREQUIL, where ASCOREQUIL is mixed with a mildew inhibitor, multivitamin, vitamins A, B and D, tocopherol and carotene, stirred with pig feed and then used to consecutively feed the boars for one week; and (3) improvement of the movement of boars and feeding with raw eggs and carrots every day.

In method (1), the testosterone used for intramuscular injection belongs to androgen drugs, thus though the injection of testosterone can increase the sexual desire of boars, long-term use can cause testicular atrophy of boars, inhibiting the sperm production. Besides, some side effects such as erythrocytosis, emesis, rash, angioneurotic edema and abnormal liver function may also occur, which also have an effect on the sperm-producing duration. As for the method (2), the main components of ASCOREQUIL are L-carnitine, aspartic acid, VB1, glutamic acid, VB6 and glycine, and although ASCOREQUIL can effectively improve the fertilization rate of livestock and poultry and reduce the mortality, it has been found that the oral liquid of ASCOREQUIL has no significant effect on the ejaculatory volume of Large White pigs and Landrace pigs. In addition, ASCOREQUIL is imported from Italy and has a relatively high price. With respect to method (3), it shows limited improvement in the sperm motility and is time-consuming.

### SUMMARY

An object of this application is to provide a method for preparing an agonist for improving boar sperm motility to overcome the defects on the prior art. The method disclosed herein has low cost, simple operation, remarkable effect and small toxic effect, by which the agonist for improving the boar sperm motility prepared involves in-vitro use, good application effect, low cost and easy production, thus having good market prospect.

The technical solutions of this application are described as follows.

This application provides a method for preparing an agonist for improving boar sperm motility, comprising:

(1) collecting a testicular tissue from a young boar 3 days after born, and infiltrating the testicular tissue in normal saline containing 1% of a double-antibody at 37° C. for 10-30 min;

(2) washing the infiltrated testicular tissue three times with PBS containing 1% of the double-antibody;

(3) incubating the testicular tissue in DMEM/F12 media at 37° C. for 30 min to fully disperse the testicular tissue, and transferring the dispersed testicular tissue to a centrifuge tube followed by centrifugation to remove a supernatant;

(4) adding 1.5 mL of hyaluronidase and 1.5 mL of collagenase IV to the centrifuge tube; shaking the centrifuge tube violently at 37° C. every other 2 min; after continuously shaken 2-3 times, adding 3 mL of DMEM/F12 media; and centrifuging the centrifuge tube to remove a supernatant;

(5) adding 1.5 mL of trypsin and 1.5 mL of deoxyribonuclease into the centrifuge tube; shaking the centrifuge tube violently at 37° C. every other 2 min; after continuously shaken 2-3 times, adding 3 mL of DMEM/F12 media to the centrifuge tube to produce a first cell suspension;

(6) filtering the first cell suspension sequentially by 80-mesh, 200-mesh, and 300-mesh cell sieves;

(7) centrifuging the filtered first cell suspension to remove a supernatant; adding 4-6 mL of a cell culture medium to form a second cell suspension; mixing the second cell suspension by blowing; transferring the second cell suspension to a cell culture dish to culture for 2 h; and replacing the cell culture medium;

(8) passaging the cells cultured in the cell culture dish; and inoculating 0.5-1.5×10⁶ cells to each of cell culture dishes having a diameter of 6 cm; and

(9) taking out the cell suspensions in the cell culture dishes after culturing for 8 h, and cryopreserving the cell suspensions at −20° C.

In an embodiment, specifically, step (1) comprises steps of: anesthetizing and castrating the young boar to collect testicles; removing blood and adipose tissues with a pair of surgical scissors and cutting up the testicles; adding the cut testicular tissue to the normal saline containing 1% of the double-antibody; and culturing the testicular tissue at 37° C. and 5% CO₂ in an incubator for 10-30 min. The young boar is collected 3-5 days after born.

In an embodiment, the double-antibody in steps (1) and (2) is a mixture of penicillin (10,000 IU) and streptomycin (10,000 μg/mL) at a concentration that is 100 times the working concentration.

The DMEM/F12 media in steps (3) and (4) is prepared by mixing F12 medium and DMEM in a ratio of 1:1, and it is suitable for clone culture at a density.

The cell culture medium in step (7) is a RPMI-1640 medium.

In step (8), the cell concentration is 1×10⁶ cells/mL.

The double-antibody used herein is a mixture of penicillin and streptomycin, which is specially used for cell culture and can be directly introduced to the cell suspension. Concentrations of penicillin and streptomycin in the mixture are respectively 10000 U/mL and 10 mg/mL, which are 100 times the recommended working concentrations thereof for cell culture. PBS (Phosphate Buffered Saline) is used for molecular cloning and cell culture, and mainly contains potassium dihydrogen phosphate and disodium hydrogen phosphate.

The DMEM/F12 medium is prepared by mixing F12 medium and DMEM in a ratio of 1:1 and is suitable for the culture of clone density, where the F12 medium is complex in composition and contains various trace elements, and DMEM contains various amino acids and glucose, and is developed on the basis of MEM. Hyaluronidase is generic for the enzymes capable of lowering the molecular weight of hyaluronic acid, which can reduce the in-vivo activity of hyaluronic acid, improving the permeability of the liquid in tissues.

Collagenase IV is a protease, and specifically an endopeptidase, and can specifically recognize the Pro-X-Gly-Pro sequence which is found frequently in collagen but rarely in other proteins and digest the peptide bond between a neutral amino acid (X) and glycine (Gly). Many proteases are able to hydrolyze single stranded and denatured collagen peptides, but the collagenase is the only one that can digest natural collagen fibers with triple supercoiled structures which are widely present in the connective tissues.

Trypsin, as a kind of protease, is a serine protease extracted from the pancreases of cattle, sheep and pigs, and acts as a digestive enzyme in vertebrate. Trypsinogen, as the precursor of trypsin, is synthesized in the pancreas and secreted as a component of pancreatic juice, and then restrictively decomposed by enterokinase or trypsin to form an activated trypsin which is an endopeptidase capable of cleaving the carboxyl side of the lysine and arginine residues in the polypeptide chain. The endopeptidase not only acts as a digestive enzyme, but also restrictively digests precursors of other enzymes such as chymotrypsin, carboxypeptidase and phospholipase to provide an activating effect. The endopeptidase has the highest specificity, playing an indispensable role in determining the amino acid arrangement of a protein.

Deoxyribonuclease, i.e., DNase is used to remove DNA from protein samples but fails to hydrolyze the tightly-arranged nuclear chromatins.

The invention has the following beneficial effects.

The agonist prepared herein involves simple in-vitro use, low cost, remarkable effect and small toxic effect, and can effectively improve the micro-environment of the boar sperms, improving the artificial insemination. Meanwhile, the agonist of the invention for boar sperms is easy to produce and use, and has long-acting effect, suitable for wide application.

### BRIEF DESCRIPTION OF THE DRAWINGS

FIG. 1 shows the trajectory of sperms in the experimental group added with an agonist, which was observed by a computer-aided analysis system.

FIG. 2 shows the trajectory of sperms in the control group added with normal saline of the same volume as agonist, which was observed by the computer-aided analysis system.

### DETAILED DESCRIPTION OF EMBODIMENTS

The invention will be further described below with reference to the embodiments. Described below are merely preferred embodiments of the invention and are not intended to limit the invention. Those skilled in the art may obtain other equivalent embodiments based on the technical solutions disclosed above. Any variations and modifications made without departing from the spirit of the invention should fall within the scope of the invention.

### EXAMPLE 1

This example provided a method for preparing an agonist for improving boar sperm motility, as described below.

(1) A testicular tissue was collected from a young boar 3-5 days after born, and then infiltrated in normal saline containing 1% of a double-antibody at 37° C. for 10-30 min.

(2) The infiltrated testicular tissue was washed three times with PBS containing 1% of the double antibody.

(3) The testicular tissue was incubated in DMEM/F12 media at 37° C. for 30 min to fully disperse the testicular tissue. Then the dispersed testicular tissue was transferred to a centrifuge tube and centrifuged to remove a supernatant.

(4) The centrifuge tube was added with 1.5 mL of hyaluronidase and 1.5 mL of collagenase IV and shaken violently at 37° C. every other 2 min. After continuously shaken 2-3 times, the centrifuge tube was added with 3 mL of DMEM/F12 media and centrifuged to remove a supernatant.

(5) The centrifuge tube was added with 1.5 mL of trypsin and 1.5 mL of deoxyribonuclease and shaken violently at 37° C. every other 2 min. After continuously shaken 2-3 times, the centrifuge tube was added with 3 mL of DMEM/F12 media to produce a first cell suspension.

(6) The first cell suspension was filtered sequentially by 80-mesh, 200-mesh and 300-mesh cell sieves.

(7) The filtered cell suspension was centrifuged to remove a supernatant, and the cells were added with 4-6 mL of a cell culture medium to form a second cell suspension. The second cell suspension was mixed by blowing, and transferred to a cell culture dish to culture for 2 h, and then the cell culture medium was replaced.

(8) The cells in the cell culture dish were passaged and inoculated at 1×10⁶ cells/per cell culture dish having a diameter of 6 cm.

(9) The cell suspension was transferred from the cell culture dish after cultured for 8 h and then cryopreserved at −20° C.

### EXAMPLE 2

This example provided a method for preparing an agonist for improving boar sperm motility, as described below.

(1) A testicular tissue was collected from a young boar 3-5 days after born, and then infiltrated in normal saline containing 1% of double-antibody at 37° C. for 10-30 min.

(2) The infiltrated testicular tissue was washed three times with PBS containing 1% of the double-antibody.

(3) The testicular tissue was incubated in DMEM/F12 media at 37° C. for 30 min to fully disperse the testicular tissue. Then the dispersed tissue was transferred to a centrifuge tube and centrifuged to remove a supernatant.

(4) The centrifuge tube was added with 1.5 mL of hyaluronidase and 1.5 mL of collagenase IV and shaken violently at 37° C. every other 2 min. After continuously shaken 2-3 times, the centrifuge tube was added with 3 mL of DMEM/F12 media, and centrifuged to remove a supernatant.

(5) The centrifuge tube was added with 1.5 mL of trypsin and 1.5 mL of deoxyribonuclease and shaken violently at 37° C. every other 2 min. After continuously shaken 2-3 times, the centrifuge tube was added with 3 mL of DMEM/F12 media to produce a first cell suspension.

(6) The first cell suspension was filtered sequentially by 80-mesh, 200-mesh and 300-mesh cell sieves.

(7) The filtered cell suspension was centrifuged to remove a supernatant, and the cells were added with 4-6 mL of a cell culture medium to form a second cell suspension. The second cell suspension was mixed by blowing, and transferred to a cell culture dish to culture for 2 h, and then the cell culture medium was replaced.

(8) The cells in the cell culture dish were passaged and inoculated at 1×10⁶ cells/per cell culture dish having a diameter of 6 cm.

(9) The cell suspension was transferred from the cell culture dish after cultured for 8 h and then cryopreserved at −20° C.

In step (1), the young boar was anesthetized and castrated to collect testicles. Blood and adipose tissue were removed with a pair of surgical scissors and the testicles were cut up. The cut testicular tissue was added to the normal saline containing 1% of the double-antibody and cultured at 37° C. and 5% CO₂ in an incubator for 10-30 min. The young boar was collected 3-5 days after born.

The double-antibody was a mixture of penicillin (10000 IU) and streptomycin (10000 μg/mL).

The DMEM/F12 media was prepared by mixing F12 medium and DMEM in a ratio of 1:1, suitable for clone culture at a density.

In step (7), the cell-culture medium was RPMI-1640 medium.

In step (8), the cells were inoculated to the cell culture dish at an amount of 1×10⁶.

It should be noted that all the instruments used herein must be sterilized under high temperature and pressure before use (121° C., 20-30 min).

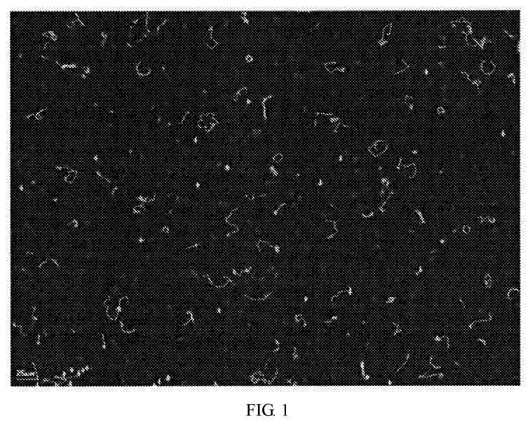

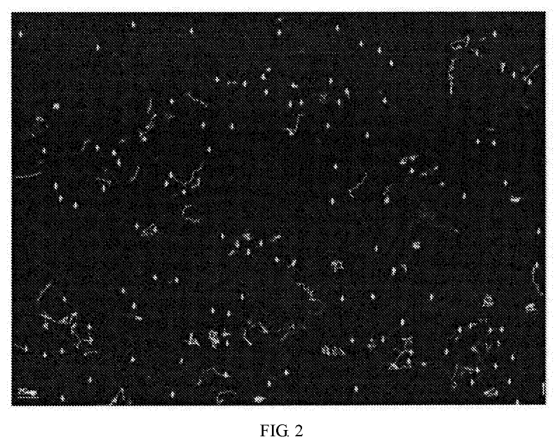

The invention could effectively improve the sperm motility of the boar, specifically, the invention castrated a young boar aged 3-5 days to collect testicular tissue cells, and the sperm motility was found to be improved by changing the micro-environment. The experimental results showed that the invention could effectively improve the sperm motility of the boars by more than 18.5%. Since the sperm motility of the boar was improved, the industrial production cost was reduced and the value of the boar and the sperm thereof was improved. The motion states of boar sperms in the experimental and control groups observed by the computer-aided analysis system were respectively shown in FIGS. 1 and 2. Described above are merely preferred embodiments of the invention. It should be noted that those skilled in the art can make various improvements without departing from the spirit of the invention and these improvements should be considered to fall within the scope of the invention.

The effect of the new in-vitro agonist for boar sperms of the invention is further described with reference to the following embodiments.

### EXAMPLE 3

### Drug Test

Young boars aged 3-5 days were selected from a pig farm at Laixi (Qingdao, Shandong), and healthy young boars were randomly selected and castrated to collect testicular tissues. The agonists prepared in Examples 1-2 of the invention were applied. 4 boars (respectively labeled as Nos. 1, 2 and 3 and control) at the estrus were selected randomly and treated by artificial vagina to collect semen samples. The semen samples were placed in a clean and sterile centrifuge tube and cryopreserved. In the experiment, the cryopreserved boar semen was examined by a microscope for the sperm motility and the motion rate was recorded. Semen samples of the boars 1-3 were respectively added with the agonist in a volume ratio of 10:1 and fully shaken. The semen sample of the control boar was added with normal saline of the same volume and fully shaken. The sperm motion was observed under a microscope and the sperm motility rate of respective boars was calculated using a computer-aided analysis system, and the results were shown in Table 1.

The sperm motility rate referred to a ratio of the movable sperms to all sperms in the microscopic field.

The sperms were graded as follows:

class A: moving forward fast;

class B: moving forward slowly; and

class C: immobile or moving slightly.

TABLE 1
Statistics of sperm movement

| Group | No. 1 | No. 2 | No. 3 | Control |
|---|---|---|---|---|
| Motility rate (%) | 87 | 95 | 83 | 64 |
| Proportion of A-grade sperm (%) | 79 | 82 | 73 | 11 |
| Proportion of B-grade sperm (%) | 8 | 13 | 10 | 9 |
| Proportion of C-grade sperm (%) | 13 | 5 | 17 | 7 |

It can be seen from Table 1 that the motility of the boar sperms treated with the agonist of the invention was significantly better than that of the boar sperms in the control group, indicating that agonist in vitro can significantly improve the sperm motility.

### EXAMPLE 4

### Drug Test

Young boars, aged 3-5 days, were selected from a pig farm at Pingdu (Qingdao, Shandong), and healthy young boars were selected randomly and castrated to collect testicular tissues. The agonists prepared in Examples 1-2 of the invention were used. 5 boars (respectively labeled as Nos. 1, 2, 3 and 4 and control) at the estrus were selected randomly and treated by artificial vagina to collect semen samples. After examined by a microscope for the sperm motility, the semen samples were then respectively placed in a clean and sterile centrifuge tube and cryopreserved. In the experiment, the boar 1 was subcutaneously injected with testosterone; the boar 2 was injected subcutaneously with the same dose of normal saline, and fed with an appropriate amount of ASCOREQUIL; and boars 3 and 4 were subcutaneously injected with the same dose of normal saline, and the boar 3 was treated by intensive exercise and artificially fed with raw eggs and carrots. One week later, the above five boars were treated by artificial vagina to collect respective semen samples, where the semen sample from the boar 4 was added with the agonist obtained in Example 1 or 2 in a volume ratio of 10:1, and shaken well. The semen samples of the boars 1-3 and 5 were respectively added with the same volume of normal saline and shaken well. The sperm status was observed by a microscope and the sperm motility rate of respective boars was calculated using a computer-aided analysis system, and the results were shown in Table 2.

Motility rate referred to a ratio of movable sperms to all the sperms in the microscope field.

TABLE 2
Statistics of sperm movement

| | | | | | Control |
|---|---|---|---|---|---|
| Group | No. 1 | No. 2 | No. 3 | No. 4 | No. 5 |
| Motility rate at the beginning of | 73 | 69 | 77 | 74 | 71 |
| treatment (%) | | | | | |
| Motility rate after one week of | 81 | 72 | 79 | 90 | 69 |
| treatment (%) | | | | | |
| Motility rate variation (%) | +8 | +3 | +2 | +16 | −2 |

It can be seen from Table 2 that after the subcutaneous injection of testosterone, the sperm motility rate of the boar 1 was increased by 8%; after fed with an appropriate amount of ASCOREQUIL, the boar 2 was improved by 3% in the sperm motility rate; after treated by intensive exercise and artificial feeding of raw eggs and carrots, the boar 3 was improved by 2% in the sperm motility rate; after the semen sample of the boar 4 was added with the agonist, the sperm motility rate was increased by 16%; and the sperm motility rate of the control boar 5 was lowered by 2%. The above results indicated that the motility of the sperms treated with the agonist of the invention was significantly better than that of the sperms in other experimental groups and the control group, demonstrating the significant effect of the agonist of the invention in improving sperm motility in vitro.

What is claimed is:

1. A method for preparing an agonist for improving boar sperm motility, comprising:
(1) collecting a testicular tissue from a young boar 3 days after born, and infiltrating the testicular tissue in normal saline containing 1% of a double-antibody at 37° C. for 10-30 min;
(2) washing the infiltrated testicular tissue three times with PBS containing 1% of the double-antibody;
(3) incubating the testicular tissue in DMEM/F12 media at 37° C. for 30 min to fully disperse the testicular tissue; and transferring the dispersed testicular tissue to a centrifuge tube followed by centrifugation to remove a supernatant;
(4) adding 1.5 mL of hyaluronidase and 1.5 mL of collagenase IV to the centrifuge tube; shaking the centrifuge tube violently at 37° C. every other 2 min; after continuously shaken 2-3 times, adding 3 mL of DMEM/F12 media; and centrifuging the centrifuge tube to remove a supernatant;
(5) adding 1.5 mL of trypsin and 1.5 mL of deoxyribonuclease to the centrifuge tube; shaking the centrifuge tube violently at 37° C. every other 2 min; after continuously shaken 2-3 times, adding 3 mL of DMEM/F12 media to the centrifuge tube to produce a first cell suspension;
(6) filtering the first cell suspension sequentially by 80-mesh, 200-mesh and 300-mesh cell sieves;
(7) centrifuging the filtered first cell suspension to remove a supernatant; adding 4-6 mL of a cell culture medium to form a second cell suspension; mixing the second cell suspension by blowing; transferring the second cell suspension to a cell culture dish to culture for 2 h; and replacing the cell culture medium;
(8) passaging the cells cultured in the cell culture dish; and inoculating 1×10⁶ cells to each of cell culture dishes having a diameter of 6 cm; and
(9) taking out the cell suspensions in the cell culture dishes after cultured for 8 h, and cryopreserving the cell suspensions at −20° C.

2. The method of claim 1, wherein step (1) comprises:
anesthetizing and castrating the young boar to collect testicles;
removing blood and adipose tissues with a pair of surgical scissors and cutting up the testicles;
adding the cut testicular tissue to the normal saline containing 1% of the double-antibody; and
culturing the testicular tissue at 37° C. and 5% CO₂ in an incubator for 10-30 min.

3. The method of claim 2, wherein the young boar is collected 3-5 days after born.

4. The method of claim 1, wherein the double-antibody is a mixture of penicillin (10000 IU) and streptomycin (10000 μg/mL).

5. The method of claim 1, wherein the DMEM/F12 media is prepared by mixing F12 medium and DMEM in a ratio of 1:1 for clone culture at a density.

6. The method of claim 1, wherein in step (7), the cell culture medium is a RPMI-1640 medium.

7. The method of claim 1, wherein the cells are inoculated at a concentration of 1×10⁶ cells/per cell culture dish of 6 cm in diameter.
